(19) United States
(12) Patent Application Publication
(10) Pub. No.: US 2025/0242162 A1
(43) Pub. Date: Jul. 31, 2025

(54) DELIVERY SYSTEM FOR ACTIVE-FIXATION LEAD DEPLOYMENT

(71) Applicant: Medtronic, Inc., Minneapolis, MN (US)

(72) Inventor: Amy E. Thompson-Nauman, Ham Lake, MN (US)

(21) Appl. No.: 19/033,707

(22) Filed: Jan. 22, 2025

Related U.S. Application Data
(60) Provisional application No. 63/625,857, filed on Jan. 26, 2024.

(51) Int. Cl. A61N 1/372; A61N 1/05; A61N 1/362

(52) U.S. Cl. CPC A61N 1/372; A61N 1/05; A61N 1/362

(57) ABSTRACT

In some examples, a system includes a sheath including an elongated shaft defining a lumen. The sheath also includes a distal portion with one or more apertures in the shaft, and an implantable medical lead configured to be delivered through the sheath to an implant site in a patient. The lead includes one or more fixation mechanisms, wherein the one or more fixation mechanisms, when deployed, are configured to extend through the one or more apertures in the sheath and engage with patient tissue at the implant site.

### RELATED APPLICATIONS

This application claims the benefit of U.S. Provisional Application Ser. No. 63/625,857, filed Jan. 26, 2024, the entire contents of each of which are incorporated herein by reference.

### TECHNICAL FIELD

The present application relates generally to implantable medical leads, implant tool systems and methods for implanting medical leads.

### BACKGROUND

Implantable systems, such as pacemakers with or without cardioversion or defibrillation capabilities, may treat cardiac dysfunction, such as bradycardia, tachyarrhythmia, and heart failure. Such implantable systems may include electrical devices configured to deliver therapy via electrodes, often carried by one or more implantable medical leads. Therapy for tachyarrhythmias may include shocks and/or anti-tachycardia pacing (ATP). The implantable systems may also be configured to deliver cardiac pacing to, for example, treat bradyarrhythmia or for cardiac resynchronization therapy (CRT).

Owing to the inherent surgical risks in attaching and replacing implantable medical leads directly within or on the heart, subcutaneous implantable systems have been devised, in which the implantable system and leads are located subcutaneously outside of the thorax. It has also been proposed that the distal portion of a lead of an implantable system may be implanted within the thorax, e.g., extravascularly and/or substernally.

Implantable medical leads are also used to monitor and/or deliver therapies to tissues other than the heart. Implantable medical leads may be used to position one or more electrodes within or near target nerves, muscles, or organs to deliver electrical stimulation to such tissues. Implantable medical leads may be used to position one or more sensors within or near target tissue to monitor biological signals from such tissues. As examples, implantable medical leads may be positioned in the epidural space to deliver spinal cord stimulation, or proximate to other nerves, such as pelvic nerves or renal nerves, to deliver neurostimulation to the nerves.

### SUMMARY

This disclosure describes systems for implanting leads including active fixation elements, such as extracardiac leads including elements to fixate the lead at a position relative to the heart within the anterior mediastinum. Extracardiac locations for lead placement may include extravascular locations (i.e., outside of the vasculature and/or vasculature system) and/or locations in extracardiac vessels within the thorax (e.g., including but not limited to the internal thoracic vein (ITV), the intercostal veins, the superior epigastric vein, the azygos veins, the hemiazygos veins, and accessory hemiazygos veins). In examples, this disclosure describes systems and methods for safely implanting and securing an extracardiac implantable cardioverter defibrillator lead as close to the heart as possible to avoid high pacing thresholds and diminished electrical performance.

In one example, a system includes: a sheath comprising an elongated shaft defining a lumen, wherein the sheath includes: a distal portion including one or more apertures in the shaft; and an implantable medical lead configured to be delivered through the sheath to an implant site in a patient, wherein the lead includes: one or more fixation mechanisms, wherein the one or more fixation mechanisms, when deployed, are configured to extend through the one or more apertures in the sheath and engage with patient tissue at the implant site.

In another example, a method includes: guiding a lead of a medical device system through a sheath of the medical device system from an incision to an implant site within a patient, wherein the sheath includes: an elongated shaft defining a lumen; and a distal portion including one or more apertures in the shaft, wherein the lead includes one or more fixation mechanisms, and wherein the one or more fixation mechanisms, when deployed, are configured to extend through the one or more apertures in the sheath; pressing the sheath toward the heart of the patient; deploying the one or more fixation mechanisms through the one or more apertures to engage with patient tissue at the implant site and secure the lead in the tissue; and removing the sheath while retaining the lead.

This summary is intended to provide an overview of the subject matter described in this disclosure. It is not intended to provide an exclusive or exhaustive explanation of the systems, devices, and methods described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF DRAWINGS

FIG. 1 is a conceptual drawing illustrating a front view of a patient implanted with an example extracardiac implantable medical device system.

FIG. 2 is a conceptual drawing illustrating a transverse view of a patient implanted with an example extracardiac implantable medical device system.

FIG. 3 is a conceptual drawing illustrating an example delivery system for implanting a lead.

FIG. 4A is a conceptual drawing illustrating an example distal portion of a sheath including collinear apertures.

FIG. 4B is a conceptual drawing illustrating an example distal portion of a sheath including noncollinear apertures.

FIG. 4C is a conceptual drawing illustrating another example distal portion of a sheath including noncollinear apertures.

FIG. 5 is a conceptual drawing illustrating an example distal portion of a sheath including a fixation mechanism for a lead extending from an aperture in the sheath.

FIG. 6 is a flow chart of an example technique for implanting one or more leads within a patient.

FIG. 7A is a conceptual drawing illustrating an example distal portion of a sheath and a fixation mechanism in an undeployed state.

FIG. 7B is a conceptual drawing illustrating the example distal portion of the sheath of FIG. 7A and the fixation mechanism in a deployed state extending from an aperture in the sheath.

FIG. 7C is a conceptual drawing illustrating the example distal portion of the sheath of FIG. 7A at least partially split around the fixation mechanism in a deployed state.

Like reference characters denote like elements throughout the description and figures.

### DETAILED DESCRIPTION

In accordance with the techniques of the disclosure, a delivery system is configured to allow delivery of a lead to an implant site within a patient. Extracardiac implantable cardioverter defibrillator leads may be implanted in the substernal space of a patient. In order to enhance procedural safety, a tunneling tool may be directed upward toward the sternum during tunneling, prior to lead delivery. The lead may then be implanted close to the sternum with the intervening mediastinum separating the lead and the heart.

Distance between the lead and the heart can cause sensation for the patient during pacing therapy, as the pacing therapy may need to be strong in order to overcome the resistance of tissue to deliver therapy into the cardiac tissue Furthermore, the energy required to provide effective therapy to the patient may increase with distance between the lead and the heart, which may reduce longevity of the device delivering therapy via the lead. The distance may also make pacing capture more difficult as current is scattered through patient tissue or fluids between the electrodes and the heart.

This disclosure describes systems and methods for safe implantation and securement of an extracardiac ICD lead that may improve procedural safety and closeness of the lead to the heart. In some examples, a system for delivering a lead to an implant site includes a sheath with an elongated shaft defining a lumen. A distal portion of the shaft may include one or more apertures and one or more structural weaknesses. The one or more structural weaknesses may be along a length of the distal portion.

The system may also include the implantable medical lead, configured to be delivered through the lumen of the sheath to the implant site. The lead may include one or more fixation mechanisms, wherein the one or more fixation mechanisms, when deployed are configured to extend through the one or more apertures in the shaft of the sheath and engage with patient tissue at the implant site. The one or more fixation mechanisms may further be configured to pull the lead and sheath closer to the implant site. The one or more structural weaknesses may allow the distal portion of the sheath to tear or split, further allowing the sheath to be withdrawn from the body while the one or more fixation mechanisms anchor the lead at the implant site.

In this way, during implantation of the medical lead, the systems of this disclosure may allow the lead to be implanted closer to the patient's heart tissue than previous systems, without decreasing procedural safety, thereby reducing cost and felt sensations of pacing therapy, as well as increasing longevity and reliability of the pacing therapy.

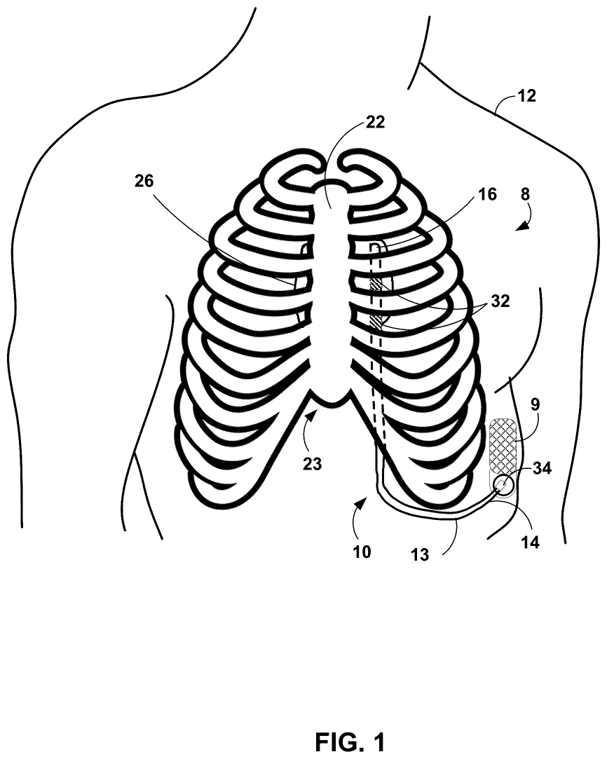

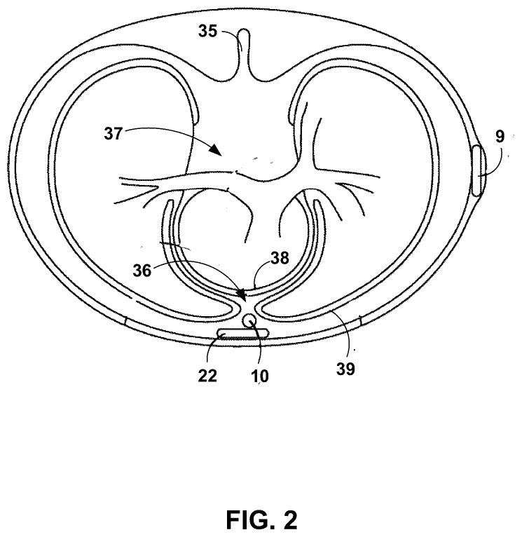

FIG. 1 is a front view of a patient 12 with an implantable medical device (IMD) system 8 implanted intrathoracically. Referring to the drawings in which like reference designators refer to like elements, FIGS. 1-2 show conceptual diagrams illustrating various views of an example extracardiac IMD system 8 demonstrating how an implantable lead may be implanted. IMD system 8 includes an IMD 9 connected to lead 10. Lead 10 may be an implantable medical lead. FIG. 1 is a front view of a patient implanted with extracardiac IMD system 8. FIG. 2 is a transverse view of patient 12 implanted with extracardiac IMD system 8.

IMD 9 may include a housing that forms a hermetic seal that protects components of the IMD 9. The housing of IMD 9 may be formed of a conductive material, such as titanium or titanium alloy, which may function as a housing electrode (sometimes referred to as a can electrode). In some examples, IMD 9 may be formed to have or may include a plurality of electrodes on the housing. IMD 9 also includes a connector assembly (also referred to as a connector block or header) that includes electrical feedthroughs through which electrical connections are made between conductors of lead 10 and electronic components included within the housing of IMD 9. As will be described in further detail herein, the housing may house one or more processors, memories, transmitters, receivers, sensors, sensing circuitry, therapy circuitry, power sources and other appropriate components. The housing may be configured to be implanted in a patient, such patient 12. Alternatively, the housing may be external to the patient and connect to a proximal end of the lead that extends out of the body of the patient, e.g., through an incision.

IMD 9 is implanted extrathoracically on the left side of the patient, e.g., under the skin and outside the ribcage (subcutaneously or submuscularly). IMD 9 may, in some instances, be implanted between the left posterior axillary line and the left anterior axillary line of the patient. IMD 9 may, however, be implanted at other extrathoracic locations on the patient, implanted in an intrathoracic location, or not implanted at all in the case of an external pacemaker.

An IMD, such as IMD 9, is coupled to lead 10. Lead 10 may be configured to be placed at a plurality of extracardiac locations, including extravascular locations (i.e., outside of the vasculature and/or vasculature system) and/or locations in extracardiac vessels within the thorax. Lead 10 may be sized to be implanted in an extracardiac location proximate the heart, e.g., intrathoracically, as illustrated in FIGS. 1-2, or extrathoracically. For example, lead 10 may extend extrathoracically under the skin and outside the ribcage (e.g., subcutaneously, submuscularly, and/or supradiaphragmatically) from IMD 9 toward the center of the torso of the patient, for example, toward the xiphoid process 23 of the patient. At a position proximate xiphoid process 23, the lead body 13 may bend or otherwise turn and extend superiorly. The bend may be pre-formed and/or lead body 13 may be flexible to facilitate bending.

In the example illustrated in FIGS. 1-2, lead body 13 extends superiorly intrathoracically anterior to heart 26 and posterior to sternum 22. In this manner, distal portion of lead 10 may reside in an extravascular intrathoracic anterior portion of the mediastinum. In some examples, lead 10 may be the only lead implanted in a patient, and lead 10 may be positioned in an anterior mediastinum 36 to achieve anterior mediastinal pacing, sensing, and/or shock therapy when the anterior mediastinum 36 is avoided and/or inaccessible for electrode implantation. In some examples, system 8 may include only a single lead positioned in an anterior mediastinum 36, a posterior mediastinum 37, or a pleural cavity of patient 12. In some examples, locations may include, but are not limited to, the pleural sac or pleural cavity, the pericardial or epicardial regions, or some intrathoracic, non-cardiac/extracardiac vascular locations. In some examples, locations may include a subcutaneous location. In some examples, locations may include a parasternal location. Although the example of FIG. 1 illustrates a length of lead 10 along a path that goes underneath xiphoid process 23, in some examples, a length of lead 10 may follow a path between the ribs (an intercostal path). For example, an access point may include an incision made on the skin/tissue of patient 12 in an intercostal location (e.g., a parasternal or apical location). Distal portion 16 of lead 10 may be delivered through the access point and through the ribs to anterior mediastinum 36.

Anterior mediastinum 36 may be viewed as being bounded laterally by pleurae 39, posteriorly by pericardium 38, and anteriorly by the sternum 22. In some instances, the anterior wall of anterior mediastinum 36 may also be formed by the transversus thoracis and one or more costal cartilages. Anterior mediastinum 36 includes a quantity of loose connective tissue (such as areolar tissue), adipose tissue, some lymph vessels, lymph glands, substernal musculature (e.g., transverse thoracic muscle), the thymus gland, branches of the internal thoracic artery, and the internal thoracic vein (ITV). Posterior mediastinum 37 may be viewed as being bounded laterally by pleurae 39, posteriorly by vertebral column 35, and anteriorly by pericardium 38. Posterior mediastinum 37 includes part of the descending aorta, the azygos and the two hemiazygos veins, the vagus and splanchnic nerves, the esophagus, the thoracic duct, and some lymph glands. The mediastinum may be bounded inferiorly by the diaphragm.

Lead body 13 may be formed from a non-conductive material, including silicone, polyurethane, fluoropolymers, mixtures thereof, and other appropriate materials, and shaped to form one or more lumens (not shown), however, the techniques are not limited to such constructions. The distal portion 16 may be fabricated to be biased in a desired configuration, or alternatively, may be manipulated by the user into the desired configuration. For example, distal portion 16 may be composed of a malleable material such that the user can manipulate distal portion 16 into a desired configuration where it remains until manipulated to a different configuration.

Lead body 13 may include a proximal end 14 having a connector 34 configured to couple to IMD 9 and a distal portion 16 which include electrodes configured to deliver electrical energy to the heart or sense electrical signals of the heart. The connectors of lead bodies 13 may be industry standard connectors (e.g., IS1, DF1, IS4, DF4, or the like) or propriety connectors. In some instances, distal portion 16 may be anchored to a desired position within the patient, for example substernally by suturing the distal portions to the patient's musculature, tissue, or bone at the xiphoid process entry site. In some examples, distal portion 16 may be anchored to the patient or through the use of one or more fixation mechanisms, e.g., rigid tines, prongs, barbs, clips, screws, and/or other projecting elements or flanges, disks, pliant tines, flaps, porous structures such as a mesh-like elements and metallic or non-metallic scaffolds that facilitate tissue growth for engagement, bio-adhesive surfaces, and/or any other non-piercing elements.

Distal portion 16 may include one or more electrodes configured to deliver therapy to heart 26 of patient 12. In some examples, one or more of the fixation mechanisms may also serve as one or more of the electrodes of the lead. That is, one or more of the fixation mechanisms may be electrically active to serve as pacing electrodes. In some examples, one or more electrodes of the lead are positioned along a length of the lead in close proximity with the one or more fixation mechanisms in order to draw the electrodes close to target tissue when the one or more fixation mechanisms are deployed at the target site. In some examples, distal portion 16 includes a plurality of electrodes spaced a distance apart from each other along the length of distal portion 16. In the example illustrated by FIGS. 1-2, distal portion 16 of lead 10 includes electrodes 32. In some examples, the plurality of electrodes may include at least one electrode located over the ventricular center of mass with respect to the cardiac silhouette when lead 10 is fixed at the target site. The plurality of electrodes may also include at least one electrode proximal to the previous electrode by one to five centimeters. In some examples, the plurality of electrodes may include one or more electrodes near the tip of lead 10.

Electrodes 32 may be disposed around or within the lead body 13 of distal portion 16, or alternatively, may be embedded within the wall of lead body 13. In one configuration, electrodes 32 may be coil electrodes formed by a conductor. The conductor may be formed of one or more conductive polymers, ceramics, metal-polymer composites, semiconductors, metals or metal alloys, including but not limited to, one of a combination of the platinum, tantalum, titanium, niobium, zirconium, ruthenium, indium, gold, palladium, iron, zinc, silver, nickel, aluminum, molybdenum, stainless steel, MP35N, carbon, copper, polyaniline, polypyrrole, and other polymers. The coil electrodes may have a surface area and/or other properties configured for delivery of antitachyarrhythmia shocks, such as defibrillation shocks, or other relatively high energy electrical therapy.

In addition to or instead of electrodes 32 configured for delivery of antitachyarrhythmia shocks, lead 10 may include electrodes 32 configured to deliver cardiac pacing or other relatively lower energy electrical therapy to a right atrium (RA), a right ventricle (RV), and/or a left ventricle (LV) of heart 26 of patient 12. One or more electrodes 32 may be configured to deliver pacing pulses to heart 26 and/or sense electrical activity of heart 26. Such electrodes may be referred to as pacing electrodes, sensing electrodes, or pace/sense electrodes.

The housing of IMD 9 may house one or more processors, memories, transmitters, receivers, sensors, sensing circuitry, therapy circuitry, power sources (e.g., capacitors and batteries), and/or other components. The components of IMD 9 may generate and deliver electrical therapy such as anti-tachycardia pacing, cardioversion or defibrillation shocks, post-shock pacing, bradycardia pacing, and/or CRT.

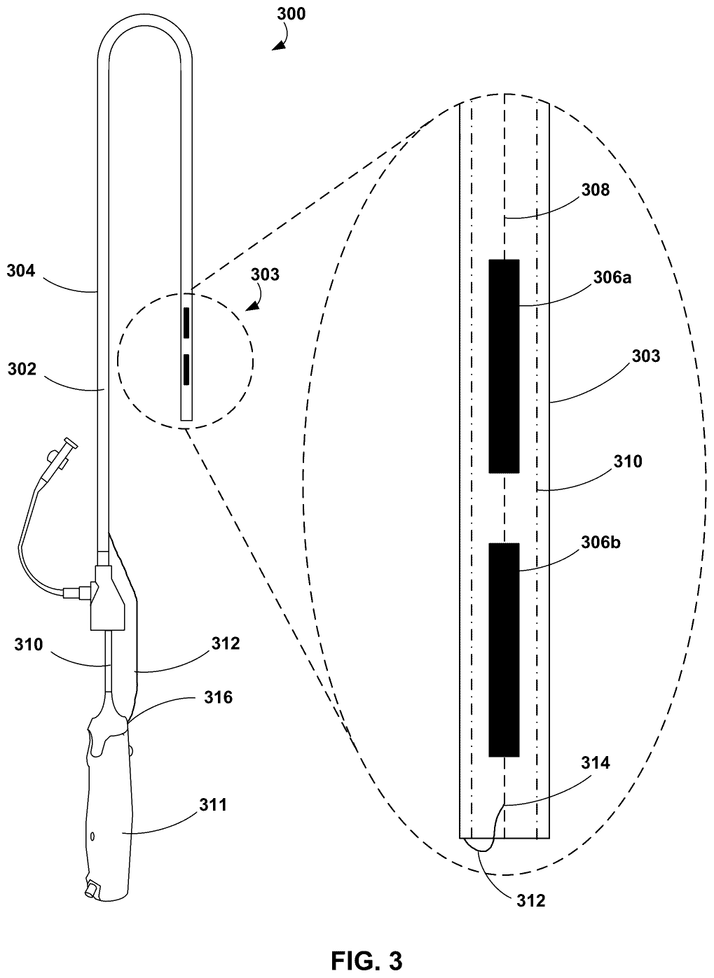

FIG. 3 is a conceptual drawing illustrating an example delivery system 300 for implanting a lead 310. System 300 may include at least hub 311, sheath 302, and pull wire 312. Sheath 302 includes elongated shaft 304 defining a lumen, wherein lead 310 of system 300, e.g., lead 10 of FIGS. 1-2, is guided through the lumen of elongated shaft 304 to an implant site. The implant site may be a target tissue site for therapy by electrodes of lead 310 of an IMD system, e.g., lead 10 of IMD system 8 of FIGS. 1-2. Sheath 302 also includes distal portion 303 with aperture 306a and aperture 306b (together, apertures 306 or one or more apertures 306). Distal portion 303 may further include one or more structural weaknesses 308 along a length of distal portion 303.

Hub 311 may include a grip and other features to be manipulated by a user of system 300 in order to control the components of system 300 during implantation. Pull wire 312 may include first, proximal end 316, and second distal end 314. The second, distal end 314 may be attached to distal portion 303 of sheath 302. The first, proximal end 316 of pull wire 312 may be configured to be disposed outside the patient's body when sheath 302 is fully inserted in the patient's body. For example, pull wire 312 may have a length sufficient to extend from distal end 314 of pull wire 312 out of the incision site in patient tissue when sheath 302 is fully inserted in the patient (e.g., when distal portion 303 is located at the target site). In some examples, proximal end 316 of pull wire 312 may attach to hub 311, and allow a user of system 300 to apply a force to pull wire 312 via hub 311. In some examples, proximal end 316 of pull wire 312 may not attach to hub 311, but be disposed in close proximity to the hub and allow a user of system 300 to apply a force to pull wire 312 using a different tool, e.g., their fingers. In some examples, proximal end 316 of pull wire 312 may be disposed outside of the incision site, whether in close proximity to hub 311 or not, in order to allow a user to apply a force to pull wire 312 using another tool and/or fingers. Although distal end 314 of pull wire 312 is depicted in FIG. 3 attached to distal portion 303 near a proximal end of distal portion 303, in some examples, distal end 314 of pull wire 312 may be attached to distal portion 303 anywhere along the length of distal portion 303. For example, distal end 314 of pull wire 312 may attach to the distal end of distal portion 303. Although the second end of pull wire 312 is herein also described as the “distal” end, in some examples, as described below, pull wire 312 may follow a path that loops around the distal end tip of sheath 302 and traverses back along a length of sheath 302 in a proximal direction before attaching to sheath 302 at the second end (distal end 314) of pull wire 312, such that the second end (distal end 314) is not the most distal portion of pull wire 312, but is still distal compared to the first, proximal end 316.

In order to implant an IMD system such as IMD system 8 of FIGS. 1-2, delivery system 300 may include other components not shown in FIG. 3. For example, system 300 may include a tunneling rod configured to create a tunneling path in a substernal location of the patient. The tunneling rod may be inserted into the patient along with sheath 302. Once tunneling is complete, the tunneling rod may be removed while retaining sheath 302 inside the patient. An implantable medical lead 310 with electrodes on a distal portion of lead 310 may be inserted into sheath 302 and guided to the implant site. Once a distal portion of lead 310 is in close proximity with the implant site, one or more fixation mechanisms of lead 310 may be deployed into patient tissue to engage with the patient tissue and draw the electrodes closer to the target tissue for therapy.

Sheath 302 may comprise a flexible, biocompatible material such as, for example, silicone or polyurethane. In some examples, sheath 302 may further include a radiopaque marker to facilitate fluoroscopic or other visualization of sheath 302, e.g., for steering and/or orienting sheath 302, as it is being delivered to the implant site. In some examples, sheath 302 may be a steerable sheath. That is, in some examples, sheath 302 may include features that allow it to effectively transfer force applied to a proximal end of sheath 302, e.g., via hub 311, into motion of a distal end of sheath 302. Such mechanisms may include reinforcement and/or pull wires, as examples. In some examples, sheath 302 may be a guidable sheath and include a lumen for receiving a guide wire to assist with advancing the sheath 302 into a desired position within the patient.

Sheath 302 includes distal portion 303 configured to be proximate one or more implant sites within the patient. For example, distal portion 303 may comprise a portion of a length of sheath 302 near a distal end of sheath 302 such that, when fully inserted into the patient, distal portion 303 is proximate one or more implant sites within the patient. In some examples, distal portion 303 may be defined by the portion of sheath 302 that is proximate to the one or more implant sites when sheath 302 is fully inserted into the patient. Distal portion 303 also includes one or more apertures 306 configured to allow one or more fixation mechanisms of lead 310 of system 300 to extend from within the lumen defined by elongated shaft 304 outside of sheath 302.

When implanting a lead for therapy inside a patient, a delivery system may not be able to position a distal portion of the lead directly adjacent to the target tissue for therapy. Distance between the lead and the target tissue for therapy may require higher energy stimulation from the electrodes in order to accomplish the proper level of therapy in the target tissue. The higher stimulation energy may cause sensation for a patient, as the patient may be able to feel the stimulation in tissues that are not the target for therapy. The higher stimulation energy may also require more power to provide therapy, reducing the battery life of a therapy delivery system. Higher stimulation energy may also reduce the useful lifespan of the therapy delivery system. Finally, the distance between the lead and the target tissue for therapy may make therapy intermittent or inconsistent, as stimulation energy is scattered through intermediate patient tissue or fluids between the electrodes and the target tissue.

These issues may be especially aggravated in therapy systems that include electrodes along a length of a distal portion of the lead, rather than only at a distal tip of the lead. For example, while a distal tip of the lead may be positioned properly adjacent target tissue, the pliable nature of the lead may allow electrodes along the length of the lead away from the distal tip to drift away from the target tissue.

Delivery system 300 includes apertures 306 configured to allow one or more fixation mechanisms of lead 310 of system 300 to engage with patient tissue while lead 310 is disposed within the lumen defined by elongate body 304 of sheath 302. For example, apertures 306 may include one or more holes in the body of sheath 302 sized to allow the one or more fixation mechanisms, when deployed, to extend through apertures 306 and engage with patient tissue at the implant site. In the example of FIG. 3, the one or more fixation mechanisms are in a non-deployed state. Apertures 306 may be disposed along a length of sheath 302 in positions that correlate to the positions of fixation mechanisms along a length of the implantable lead. Furthermore, apertures 306 may be disposed along the length of sheath 302 in positions that correlate to the one or more target implant sites when sheath 302 is fully inserted into the patient.

In some examples, one or more markers may be located along the length of sheath 302 to indicate a positioning of sheath 302 within the patient. In some examples, one or more markers may be located in a proximal portion of sheath 302 at a location outside the patient's body when sheath 302 is fully inserted into the patient, and may be visually checked with the naked eye to indicate depth and/or orientation of sheath 302. In some examples, one or more markers may be located in a proximal portion of sheath 302 at a location inside the patient's body when sheath 302 is fully inserted into the patient, and may be visually checked through radiography, fluoroscopy, or other method to indicate depth and/or orientation of sheath 302 within the patient's body. For example, the one or more markers may be radiopaque such that they easily appear on a radiograph. In some examples, the one or more markers may be located in a distal portion of sheath 302, or anywhere else along the length of sheath 302 to indicate depth and/or orientation of sheath 302 within the patient's body.

For example, apertures 306 may be spaced along the length of distal portion 303 such that when first aperture 306a is positioned at a first implant site near a first target tissue, second aperture 306b is positioned at a second implant site near a second target tissue. Lead 310 of system 300 may include a first fixation mechanism, a second fixation mechanism, a first electrode, and a second electrode. The first fixation mechanism and the first electrode may be positioned along a length of lead 310 substantially coextensive with first aperture 306a along the length of sheath 302. When deployed, the first fixation mechanism may be configured to extend through first aperture 306a, engage with the patient tissue at the first implant site, and hold first electrode close to the first implant site. The second fixation mechanism and the second electrode may be positioned along a length of lead 310 substantially coextensive with second aperture 306b along the length of sheath 302. When deployed, the second fixation mechanism may be configured to extend through second aperture 306b, engage with the patient tissue at the second implant site, and hold second electrode close to the first implant site. Although described with reference to a single fixation mechanism and a single electrode corresponding to a single aperture of apertures 306, in some example, one or more fixation mechanisms, implant sites, and/or electrodes may generally correspond to a single aperture of apertures 306. In some examples, one or more of the fixation mechanisms may also serve as one or more of the electrodes of lead 310. In some examples, one or more electrodes of lead 310 are positioned along a length of lead 310 in close proximity with the one or more fixation mechanisms.

In some examples, the one or more fixation mechanisms have a compressed state and an uncompressed state. Before deployment, the one or more fixation mechanisms may be compressed within a lumen defined by sheath 302. During deployment, the one or more fixation mechanisms may extend out of e.g., apertures 306a and/or 306b to an uncompressed state in which the one or more fixation mechanisms engage with patient tissue. The one or more fixation mechanisms may transition from the compressed state to the uncompressed state via a tool or portion of hub 311 that is manipulated by a user of system 300.

Because lead 310 of system 300 may be attached to patient tissue while lead 310 is still disposed within the lumen defined by elongate body 304, distal portion 303 of sheath 302 may need to split in order for sheath 302 to be removed from the body of the patient. For example, material of distal portion 303 may need to destructively yield so that distal portion 303 does not get stuck on the fixation mechanisms when a physician tries to remove sheath 302 from the patient's body. Therefore, system 300 includes structural weaknesses in distal portion 303 configured to allow distal portion 303 to split in response to a force. In some examples, the force may be provided by pull wire 312. In some examples, the force of the interaction between the fixation mechanisms and distal portion 303 when sheath 302 is withdrawn from the patient is sufficient to cause distal portion 303 to destructively yield along structural weaknesses 308.

Structural weaknesses 308 may include any intentional weakness in the material of distal portion 303. For example, structural weaknesses 308 may include a plurality of perforations along the length of distal portion 303. In some examples, structural weaknesses 308 may include a channel along the length of distal portion 303 where a thickness of distal portion 303 is thinner than the surrounding material. In some examples, structural weaknesses 308 may include the inclusion of a different material in distal portion 303 in a path along the length than the material that makes up the rest of distal portion 303, wherein the different material has a yield strength that is less than the yield strength of the primary material that makes up distal portion 303.

Although structural weaknesses 308 are herein primarily described as only existing in distal portion 303 of sheath 302, in some examples structural weaknesses 308 may exist along the entire length of sheath 302. For example, sheath 302 may comprise a slittable sheath with one or more perforations along the length of sheath 302. When removing sheath 302 from a patient's body, a physician may use a slitting tool to begin splitting sheath 302 at a proximal end in addition to, or in lieu of splitting sheath 302 at distal portion 303 using pull wire 312. In some examples, structural weaknesses 308 only exist in distal portion 303 of sheath 302. In some examples, distal portion 303 of sheath 302 may be defined by the distal portion of sheath 302 that includes structural weaknesses 308.

Distal end 314 of pull wire 312 may attach to distal portion 303 in order to facilitate tearing of distal portion 303. For example, distal end 314 of pull wire 312 may attach to distal portion 303 at or near structural weaknesses 308, such that when a force is applied to pull wire 312 near hub 311, at least a portion of that force is transferred through pull wire 312 to distal portion 303 of sheath 302. Sheath 302 may be configured to tear in response to the portion of the force transferred through pull wire 312. For example, the portion of the force transferred through pull wire 312 may be sufficient to cause the material of sheath 302 to yield along structural weaknesses 308. Although distal end 314 of pull wire 312 is depicted in FIG. 3 attached to distal portion 303 near a proximal end of distal portion 303, in some examples, distal end 314 of pull wire 312 may be attached to distal portion 303 anywhere along the length of distal portion 303. For example, distal end 314 of pull wire 312 may attach to the distal end of distal portion 303.

Although pull wire 312 is depicted in FIG. 3 as outside of sheath 102, in some examples, pull wire 312 may be disposed partially or entirely within a lumen defined by elongate body 304. In some examples, the lumen in which pull wire 312 is disposed may be the same lumen in which lead 310 is progressed to the implant site. In some examples, pull wire 312 may be at least partially disposed in a different lumen defined by sheath 302 than the lumen defined by elongated body in which lead 310 may be progressed to the implant site. In some examples, pull wire 312 may be disposed within a lumen defined by sheath 302 along a portion of the length of sheath 302, then exit sheath 302 to travel along the outside of sheath 302 until reaching the attachment point of pull wire 312 on distal portion 303. In some examples, pull wire 312 may first travel outside sheath 302 along a length of sheath 302 before entering a lumen defined by sheath 302 and traveling within the lumen to the attachment point of pull wire 312 on distal portion 303. In some examples, pull wire 312 may travel in and out of a lumen defined by sheath 302 any number of times.

In some examples, a pull wire 312 may run through the entire length of distal portion 303 while disposed inside sheath 302, exit the distal tip of sheath 302, and wrap back around the outside of sheath 302 before attaching to a location on the outside surface of distal portion 303. In some examples, the length of pull wire 312 from the exit of the distal tip of sheath 302 to the attachment point of pull wire 312 on distal portion 303 may follow one or more structural weaknesses 308 along the length of distal portion 303. In this manner, as a force is exerted on pull wire 312, it may act to tear distal portion 303 along structural weaknesses 308 from the distal tip proximally.

Although this disclosure is primarily described with reference to a lead implanted near the heart of a patient. It may be understood that the techniques of this disclosure are applicable to leads implanted in other areas of the body as well. For example, delivery system 300 may be used to deliver a lead for therapy to other areas of the body as well, and the systems and techniques described herein may allow the electrodes of the lead to be brought closer the target tissue site for therapy. Furthermore, although the primary therapy described herein is for cardiac pacing, in some examples, the IMD system may be configured to provide any therapy appropriate for the location of the implantation.

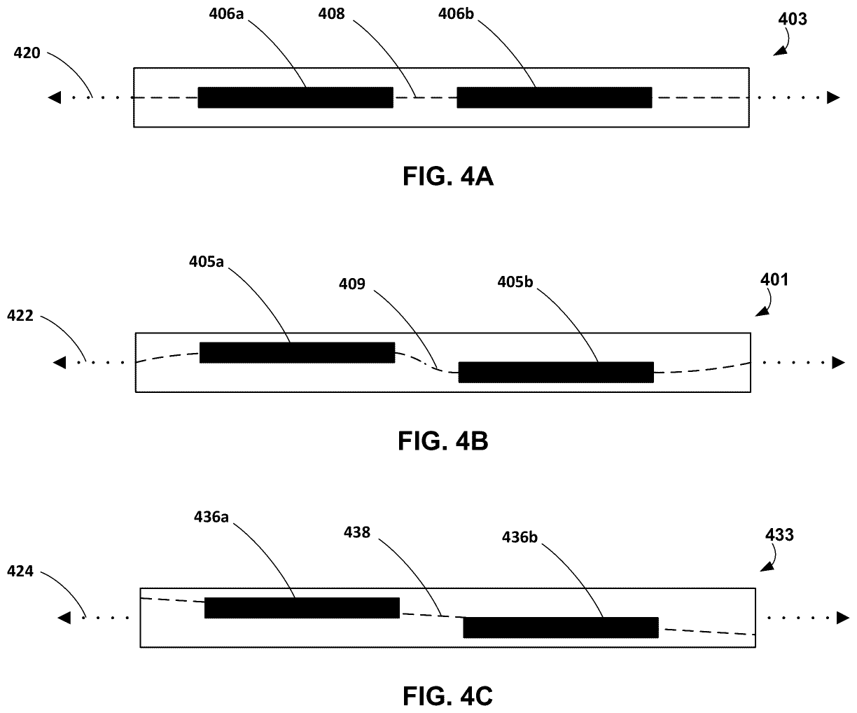

FIG. 4A is a conceptual drawing illustrating an example distal portion 403 of a sheath including collinear apertures 406. In some examples, distal portion 403 may be substantially similar to distal portion 303 from FIG. 3. In some examples, apertures 406 may be substantially similar to apertures 306 from FIG. 3. In some examples, structural weaknesses 408 may be substantially similar to structural weaknesses 308 from FIG. 3. Distal portion 403 may include a length defining a longitudinal axis 420. In the example of FIG. 4A, axis 420 is not shown along the length of distal portion 403 in order to avoid confusion with the path of structural weaknesses 408, however it may be understood that axis 420 extends through the length of distal portion 403. In the example of FIG. 4A, apertures 406 are collinearly disposed on distal portion 403 along longitudinal axis 420.

Structural weaknesses 408 may form a path along the length of distal portion 403 of the sheath, wherein the path intersects with each of apertures 406. Although only apertures 406a and 406b are depicted in FIG. 4A it may be understood that the path of structural weaknesses 408 may intersect with each of the apertures in a distal portion no matter the number of apertures. In this way, when distal portion 403 yields as a result of the applied force, the sheath may be withdrawn from the patient's body without getting stuck on deployed fixation mechanisms. For example, the deployed fixation mechanisms may pass through the slit created by the yielded material of distal portion 403 as the sheath is withdrawn from the patient's body. In the example of FIG. 4A, structural weaknesses 408 define a straight path through the length of distal portion 403 on the visible side of distal portion 403. In some examples the path of structural weaknesses 408 may be substantially parallel with longitudinal axis 420 along the length of distal portion 403. For example, structural weaknesses 408 may include perforations forming a path that is collinear along longitudinal axis 420 with apertures 406.

In some examples, apertures 406 may be collinear with one another along a line parallel to longitudinal axis 420, and structural weaknesses 408 may define a straight path parallel to longitudinal axis 420, but the path of structural weaknesses 408 may be slightly offset from the line along which apertures 406 are collinear.

FIG. 4B is a conceptual drawing illustrating an example distal portion 401 of a sheath including noncollinear apertures 405. In some examples, distal portion 401 may be substantially similar to distal portion 303 from FIG. 3. In some examples, apertures 405 may be substantially similar to apertures 306 from FIG. 3. In some examples, structural weaknesses 409 may be substantially similar to structural weaknesses 308 from FIG. 3.

Distal portion 401 may include a length defining a longitudinal axis 422. In the example of FIG. 4B, axis 422 is not shown along the length of distal portion 401 in order to avoid confusion with the path of structural weaknesses 409, however it may be understood that axis 422 extends through the length of distal portion 401. In the example of FIG. 4B, apertures 405a and 405b are noncollinearly disposed on distal portion 401 with respect to longitudinal axis 422. In the example of FIG. 4B, structural weaknesses 409 define a non-straight path through the length of distal portion 401. In some examples, structural weaknesses 409 may include perforations that form a path of any shape along the length of distal portion 401, so long as when distal portion 401 yields along structural weaknesses 409, the sheath may be withdrawn from the patient's body without becoming stuck on the fixation mechanisms.

In some examples, distal portion 401 may include apertures disposed at different radial locations around a circumference of distal portion 401. For example, a first set of one or more apertures may include apertures 405, and a second set of apertures may be disposed on the opposite side of distal portion 401. In some examples, structural weaknesses 409 may include a first set of structural weaknesses 409 defining a first path on one side of distal portion 401 (e.g., the side of distal portion 401 shown in FIG. 4B), and a second set of structural weaknesses defining a second path on another side of distal portion 401 (e.g., the side of distal portion 401 not shown in FIG. 4B). In some examples, both the first and second set of structural weaknesses may follow the same path with respect to a side plane defined by distal portion 401 (e.g., a side plane as shown in FIG. 4B). That is, the first path and the second path may be identical as viewed from the side plane.

FIG. 4C is a conceptual drawing illustrating another example distal portion 433 of a sheath including noncollinear apertures 436a and 436b. Distal portion 433 may include a length defining a longitudinal axis 424. In the example of FIG. 4C, apertures 436 are noncollinearly disposed on distal portion 433 with respect to longitudinal axis 424. In the example of FIG. 4C, axis 424 is not shown along the length of distal portion 433 in order to avoid confusion with the path of structural weaknesses 438, however it may be understood that axis 424 extends through the length of distal portion 433. In the example of FIG. 4C, structural weaknesses 438 define a straight path through the length of distal portion 433 that is not parallel with longitudinal axis 424 along the length of distal portion 433.

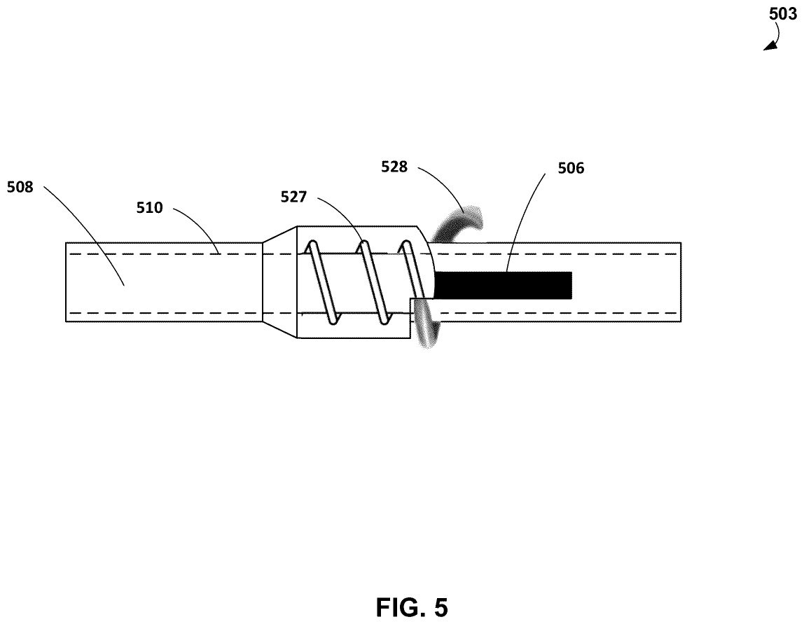

FIG. 5 is a conceptual drawing illustrating an example distal portion 503 of a sheath including fixation mechanism 528 for a lead 510 extending from aperture 506 in the sheath. In some examples, components of distal portion 503 are substantially similar to like-named components of FIGS. 1-4C. In the example of FIG. 5, fixation mechanism 528 is in an at least partially deployed state. Fixation mechanism 528 may include one or more rigid tines, prongs, barbs, clips, screws, and/or other projecting elements or flanges, disks, pliant tines, flaps, porous structures such as a mesh-like elements and metallic or non-metallic scaffolds that facilitate tissue growth for engagement, bio-adhesive surfaces, and/or any other non-piercing elements. In the example of FIG. 5, fixation mechanism includes a side helix.

Aperture 506 may be configured to allow fixation mechanism 528 to engage with patient tissue while lead 510 is disposed within the lumen defined by distal portion 503 of the sheath. For example, aperture 506 may include one or more holes in the body of distal portion 503 sized to allow fixation mechanism 528, when deployed, to extend through aperture 506 and engage with patient tissue at the implant site.

In some examples, fixation mechanism 528 may have a compressed state and an uncompressed state. Before deployment, fixation mechanism 528 may be compressed within a lumen defined by distal portion 503. In the example of FIG. 5, an uncompressed portion 527 of fixation mechanism 528 is depicted within distal portion 503 of the sheath. It may be understood that uncompressed portion 527 is shown in FIG. 5 through the outer surface of the sheath, and that uncompressed portion 527 may not be visible from outside the sheath. During deployment, fixation mechanism 528 may extend out of aperture 506 to an uncompressed state in which fixation mechanism 528 engages with patient tissue. Fixation mechanism 528 may transition from the compressed state to the uncompressed state via a tool or portion of the hub that is manipulated by a user of the system.

Fixation mechanism 528 may be configured to pull lead 510 and sheath closer to the implant site. For example, fixation mechanism 528 includes a side helix that, when deployed, engages with patient tissue. As deployment progresses, fixation mechanism 528 may pull patient tissue taught around the side helix such that one or both of the patient tissue or lead 510 is pulled towards the other. In some examples, a user may press the sheath down toward the heart so that distal portion 503 is pressed in closer proximity to target tissues before deployment of fixation mechanism 528.

After deployment of fixation mechanism 528, the sheath may be withdrawn from the patient's body. Structural weaknesses incorporated into the sheath may be configured to yield in response to a force. In some examples, the force may be provided by a pull wire as descried above. In some examples, the force of the interaction between fixation mechanism 528 and distal portion 503 as the sheath is withdrawn from the patient is sufficient to cause distal portion 503 to destructively yield along the structural weaknesses. In some examples, a physician may use a slitting tool to slit a proximal portion of the sheath and extend the slit distally along the sheath through distal portion 503, e.g., by pulling apart sheath as it is withdrawn over lead 510.

In some examples, fixation mechanism 528 may be deployed into patient tissue, and electrical testing of lead 510 may be performed before withdrawal of the sheath. For example, an electric current may be applied to and/or measured from patient tissue at the implant site to check for proper electrical response from patient tissue. If proper electrical response is not received, fixation mechanism 528 may be retracted, lead 510 and/or sheath may be repositioned within the patient, and fixation mechanism 528 may be redeployed into patient tissue at the repositioned site. This procedure may repeat until the proper electrical response is measured, indicating an effective implant location. Thereafter, fixation mechanism 528 may remain deployed at the effective implant location and the sheath may be withdrawn from the patient's body. In some examples, fixation mechanism 528 may act as an electrode of lead 510 for electrical testing purposes. In some examples, fixation mechanism 528 may not act as an electrode, but the one or more electrodes of lead 510 may be coextensive with the one or more apertures (e.g., aperture 506). In such examples, deployment of fixation mechanism 528 may pull lead 510 and the one or more electrodes toward patient tissue at the implant site such that the one or more electrodes contact patient tissue at the implant site through the one or more apertures (e.g., aperture 506), allowing electrical testing of lead 510 while the sheath is still inserted.

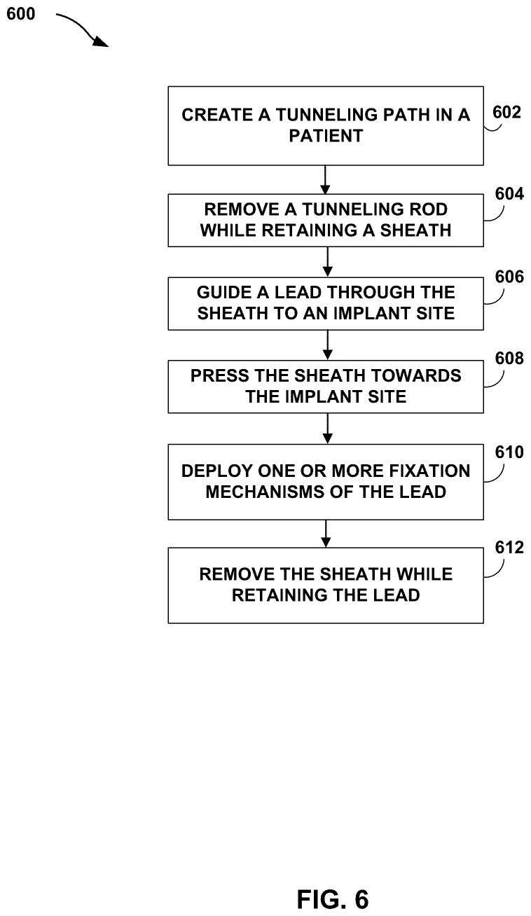

FIG. 6 is a flow chart of an example technique for implanting one or more leads within a patient. The technique of FIG. 6 may be used in connection with any of the devices or systems described in connection with FIGS. 1-5, and is described with respect to delivery system 300 of FIG. 3. The technique may be applicable for implanting one or more leads at one or more extracardiac locations, including, but not limited to, the mediastinum and/or the pleural cavity of a patient.

Technique 600 may include creating a tunneling path in a patient (602). In examples where the implant site is one or more extracardiac locations, the method may include creating an access point into the patient with an incision made on the skin/tissue of the patient, e.g., adjacent to or below the xiphoid process. In some examples, the access point may include an incision made on the skin/tissue of the patient in an intercostal location, for example, via thoracotomy (i.e., between the ribs). In some examples, the access point may include an incision proximate the manubrium. When accessing via an incision proximate the manubrium, the anterior mediastinum, the posterior mediastinum, the anterior pleural cavity, and/or the posterior pleural cavity may be accessed.

The incision may be sized to allow for insertion of a delivery tool, sheath, and/or tunneling tool of delivery system 300 and navigation of one or more leads. In some examples, more than one incision may be made to accommodate one or more leads. For example, a first incision may be made to deliver a first lead and a second incision made to deliver a second lead. The tunneling tool may be used to create the tunneling path from the incision through the patient to an implant site.

Sheath 302 may be disposed on the tunneling tool during creation of the tunneling path. The method may further include positioning the tunneling tool and sheath 302 proximate the implant site within the patient. The tunneling tool may then be removed while retaining sheath 302 within the patient (604).

Technique 600 may include guiding a lead through sheath 302 to the implant site (606). Sheath 302 may be sized to allow delivery of one or more leads through a lumen defined by sheath 302. An elongated portion of sheath 302 may be advanced within the substernal space of the patient. Sheath 302 may aid in delivering the lead at a location proximate the xiphoid process and/or an intercostal location (i.e., between the ribs).

Continuing with the example of FIG. 6. method 600 may include pressing sheath 302 and lead towards the implant site within the patient (608). For safety during creation of the tunneling path, the tunneling tool and sheath 302 may generally be directed away from the heart, e.g., toward or close to the underside of the sternum. In order to get the lead closer to the target tissue for therapy, sheath 302 and lead may be pressed towards the implant site, e.g., toward the heart. While pressing sheath 302 and lead towards the implant site, method 600 may further include deploying the one or more fixation mechanisms of the lead to engage with patient tissue at the implant site, e.g., tissue of the anterior mediastinum (610). Sheath 302 may include a distal portion with one or more apertures configured to allow the fixation mechanisms to deploy into patient tissue while the lead is disposed in a lumen of sheath 302. The deployed fixation mechanisms may attach to patient tissue and secure the lead at the implant site.

Method 600 may also include electrically testing the lead while the one or more fixation mechanisms are deployed in patient tissue. For example, method 600 may include applying to and/or measuring an electric current from patient tissue at the implant site to check for proper electrical response from patient tissue. If proper electrical response is not received, method 600 may include retracting the one or more fixation mechanisms (e.g., retracting the one or more fixation mechanism back into an interior of sheath 302), repositioning the lead and/or sheath within the patient, and redeploying the one or more fixation mechanisms into patient tissue at the repositioned site. This procedure may repeat until the proper electrical response is measured, indicating an effective implant location. Thereafter, the one or more fixation mechanisms may remain deployed at the effective implant location.

Method 600 may finally include removing sheath 302 while retaining the lead within the patient (612). For example, sheath 302 may include one or more structural weaknesses in the distal portion along 303 a length of the distal portion 303. The one or more structural weaknesses may allow the distal portion 303 of sheath 302 to tear or split in response to a force, allowing sheath 302 to be withdrawn from the body without becoming stuck on the deployed fixation mechanisms. In some examples, the force may be provided by pull wire 312. In some examples, the force of the interaction between the fixation mechanisms and distal portion 303 when sheath 302 is withdrawn from the patient is sufficient to cause distal portion 303 to destructively yield along structural weaknesses 308.

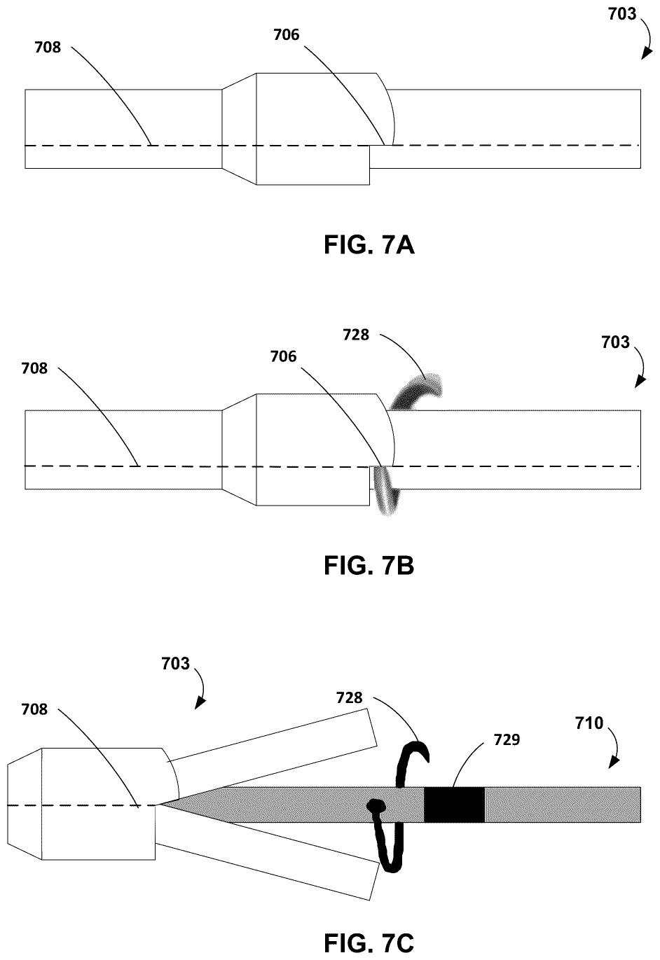

FIG. 7A is a conceptual drawing illustrating an example distal portion 703 of a sheath and fixation mechanism 728 in an undeployed state. In some examples, components of distal portion 703 in FIGS. 7A-7C are substantially similar to like-named components of FIGS. 1-5. In the example of FIGS. 7A-7C, fixation mechanism 728 includes a side helix. Distal portion 703 also includes one or more structural weaknesses 708.

Aperture 706 may be configured to allow fixation mechanism 728 to engage with patient tissue while the lead is disposed within the lumen defined by distal portion 703 of the sheath. For example, aperture 706 may include one or more holes in the body of distal portion 703 sized to allow fixation mechanism 728, when deployed, to extend through aperture 706 and engage with patient tissue at the implant site. In the example of FIG. 7A, aperture 706 may face downward relative to the figure. In an undeployed state, fixation mechanism 728 may be held within a lumen defined by distal portion 703. In the undeployed state, fixation mechanism 728 may be compressed within the lumen defined by distal portion 703.

FIG. 7B is a conceptual drawing illustrating the example distal portion 703 of the sheath of FIG. 7A and fixation mechanism 728 in a deployed state extending from aperture 706 in the sheath. In the example of FIG. 7B, fixation mechanism 528 is in an at least partially deployed state. Fixation mechanism may include a compressed and uncompressed state. As fixation mechanism 728 is deployed from aperture 706, fixation mechanism 728 may expand from a compressed state outward from the sheath to an uncompressed state in order to engage with patient tissue. The example of FIG. 7B is provided for example only, in some example, fixation mechanism 728 may extend further away from a longitudinal center of sheath 703 when fixation mechanism 728 expands to the uncompressed state. In order to deploy fixation mechanism 728, a tool or portion at the hub of the sheath may be rotated, thereby rotating fixation mechanism 728 such that it extends through aperture 706 out of the lumen defined by the sheath.

In some examples, fixation mechanism 728 may be disposed within a portion of the sheath with a larger diameter than the rest of the sheath. Aperture 706 may be located at an edge of the portion of the sheath with the larger diameter. As fixation mechanism 728 is deployed, it may extend through aperture 706 out of the larger diameter portion of the sheath and engage with patient tissue that surrounds a smaller diameter portion of the sheath.

Fixation mechanism 728 may be configured to pull the lead and sheath closer to the implant site. For example, fixation mechanism 728 includes a side helix that, when deployed, engages with patient tissue. As deployment progresses (e.g., as fixation mechanism 728 rotates), fixation mechanism 528 may further extend into patient tissue and/or pull patient tissue taught around the side helix such that one or both of the patient tissue or the lead is pulled towards the other. In some examples, fixation mechanism 728 may include features that enhance the stability of attachment to patient tissue. Although depicted in FIGS. 7B and 7C as a single prong, in some examples fixation mechanism 528 may be one of a plurality of prongs that extend into patient tissue when deployed. In some examples, fixation mechanism 728 may include a bifurcated or multifurcated tip. In some examples, fixation mechanism 728 may include a plurality of barbs along the length of fixation mechanism 728 that extends from the sheath, wherein the barbs curve or point in a direction along the length of fixation mechanism 728 away from the tip to snag on patient tissue. In some examples, fixation mechanism 728 includes a mesh or other material along the length of fixation mechanism 728 which engages with patient tissue in order to promote tissue growth ingrowth around the mesh and stabilize the attachment between fixation mechanism 728 and patient tissue.

FIG. 7C is a conceptual drawing illustrating the example distal portion 703 of the sheath of FIG. 7A at least partially split around fixation mechanism 728 in a deployed state. After fixation mechanism 728 is deployed into patient tissue at the implant site, material of distal portion 703 may need to destructively yield so that distal portion 703 does not get stuck on the fixation mechanisms when a physician tries to remove sheath. In some examples, structural weaknesses 708 may be disposed along a length of distal portion 703 intersecting with aperture 706. When distal portion 703 yields along structural weaknesses 708, the sheath may be withdrawn without distal portion 703 catching on fixation mechanism 728. In some examples, the force of removing the sheath may cause interaction between fixation mechanism 728 and an edge of aperture 706 along the path of structural weaknesses 708 that tears distal portion 703 along the path of structural weaknesses 708. In some examples, a pull wire is attached at a point of distal portion 703 along the path of structural weaknesses 708, where a force applied to the pull wire may act to tear distal portion 703 along the path of structural weaknesses 708.

Tearing distal portion 703 may reveal lead 710. Lead 720 may include one or more electrodes 729 configured to deliver therapy to a heart of the patient. In some examples, one or more fixation mechanisms (e.g., fixation mechanism 728) may also serve as one or more of the electrodes of lead 710. Electrode 729 may be positioned along a length of the lead in close proximity with fixation mechanism 728 so that, as fixation mechanism 728 engages with patient tissue and pulls lead 710 towards patient tissue, electrode 729 may be drawn close to target tissue at the target site.

In some examples, a distal portion of lead 710 includes a plurality of electrodes spaced a distance apart from each other along the length of the distal portion. For example, the plurality of electrodes may include at least one electrode located over the ventricular center of mass with respect to the cardiac silhouette when lead 710 is fixed at the target site. The plurality of electrodes may also include at least one electrode proximal to the previous electrode by one to five centimeters. In some examples, the plurality of electrodes may include one or more electrodes near the tip of lead 710. In some examples, the one or more electrodes near the tip of lead 720 may collect data from the an atrium of the patient's heart. The one or more electrodes may be disposed around or within the lead body of lead 710, or alternatively, may be embedded within the wall of the lead body.

Although aperture 706 is depicted in FIGS. 7A-7C as facing downward with respect to the figures, in some examples, aperture 706 may extend along a length of distal portion 703 coextensive with the location of electrode 729 on lead 710. In this way, electrode 729 may contact lead tissue through aperture 706 without first removing distal portion 703 of the sheath. This may allow a user to take measurements using electrode 729 to confirm proper positioning of electrode 729 and lead 710 at the implant site before removing sheath 703.

Select examples of the present disclosure include, but are not limited to, the following examples.

Example 1: A system including: a sheath including an elongated shaft defining a lumen, wherein the sheath includes: a distal portion including one or more apertures in the shaft; and an implantable medical lead configured to be delivered through the sheath to an implant site in a patient, wherein the lead includes: one or more fixation mechanisms, wherein the one or more fixation mechanisms, when deployed, are configured to extend through the one or more apertures in the sheath and engage with patient tissue at the implant site.

Example 2. The system of example 1, wherein the distal portion defines a length, and includes one or more structural weaknesses along the length configured to yield in response to a force.

Example 3. The system of example 2, wherein the force is a first force, and wherein the system further includes: a hub; a pull wire including: a first end configured to be disposed outside the patient when the sheath is fully inserted in the patient; and a second end attached to the distal portion of the sheath, wherein the pull wire is configured to transfer at least a portion of a second force applied to the pull wire at the first end to the distal portion of the sheath as the first force, and wherein the sheath is configured to tear along the one or more structural weaknesses in response to the first force.

Example 4. The system of examples 2 or 3, wherein the one or more structural weaknesses form a path along the length of the distal portion of the sheath that intersects with each of the one or more apertures.

Example 5. The system of any of examples 2-4, wherein the one or more structural weaknesses are disposed on the length of the distal portion of the sheath collinear with the one or more apertures.

Example 6. The system of any of examples 2-5, wherein the one or more structural weaknesses include one or more perforations in the distal portion of the sheath.

Example 7. The system of any of examples 1-6, wherein the one or more apertures are spaced along the length of the distal portion such that when a first aperture of the one or more apertures is positioned at the implant site, a second aperture of the one or more apertures is positioned at the implant site, wherein the one or more fixation mechanisms includes a first fixation mechanism and a second fixation mechanism, wherein the first fixation mechanism, when deployed, is configured to extend through the first aperture and engage with the patient tissue at the implant site, and wherein the second fixation mechanism, when deployed, is configured to extend through the second aperture and engage with the patient tissue at the implant site.

Example 8. The system of any of examples 1-7, wherein the implant site includes an extracardiac location.

Example 9. The system of any of examples 1-8, wherein the implant site includes a substernal location.

Example 10. The system of any of examples 1-9, wherein the implant site includes an anterior mediastinum location.

Example 11. The system of any of examples 1-10, further including an implantable medical device (IMD) configured to be coupled to the lead, wherein the one or more fixation mechanisms include one or more electrodes, and wherein the IMD is configured to deliver cardiac pacing via the one or more electrodes.

Example 12. The system of any of examples 1-11, wherein the one or more fixation mechanisms, when deployed, are configured to extend through the one or more apertures in the sheath and engage with the patient tissue while the lead remains inside the sheath.

Example 13. The system of any of examples 1-12, wherein the one or more apertures include a first aperture and a second aperture, and wherein the first aperture and second aperture are collinear along the length of the distal portion of the sheath.

Example 14. The system of any of examples 1-13, wherein the one or more apertures include a first aperture and a second aperture, and wherein the first aperture and second aperture are noncollinear along the length of the distal portion of the sheath.

Example 15. The system of any of examples 1-14, wherein the lead is configured to be positioned in an anterior mediastinum of the patient, a posterior mediastinum of the patient, or a pleural cavity of the patient.

Example 16. The system of any of examples 1-15, wherein the one or more fixation mechanisms, when deployed, are configured to pull the lead closer to the implant site.

Example 17. The system of any of examples 1-16, wherein the one or more fixation mechanisms include one or more side helices.

Example 18. The system of any of examples 1-17, wherein the one or more fixation mechanisms include one or more of side helices, rigid tines, prongs, barbs, clips, screws, disks, pliant tines, flaps, porous structures, metallic or non-metallic scaffolds, or bio-adhesive surfaces.

Example 19. A method including: guiding a lead of a medical device system through a sheath of the medical device system from an incision to an implant site within a patient, wherein the sheath includes: an elongated shaft defining a lumen; and a distal portion including one or more apertures in the shaft, wherein the lead includes one or more fixation mechanisms, and wherein the one or more fixation mechanisms, when deployed, are configured to extend through the one or more apertures in the sheath; pressing the sheath toward the heart of the patient; deploying the one or more fixation mechanisms through the one or more apertures to engage with patient tissue at the implant site and secure the lead in the tissue; and removing the sheath while retaining the lead.

Example 20. The method of example 19, further including: creating a tunneling path in a substernal location of the patient via a tunneling rod and the sheath of a medical device system; and removing the tunneling rod from the tunneling path while retaining the sheath within the tunneling path.

Example 21. The method of any of examples 19-20, wherein removing the sheath includes splitting the distal portion of the sheath along one or more structural weaknesses within the distal portion.

Example 22. The method of example 19, wherein splitting the distal portion of the sheath along the one or more structural weaknesses includes applying a force to a first end of a pull wire of the medical device system, wherein the first end is configured to be disposed outside the patient when the sheath is fully inserted in the patient, wherein the pull wire is attached at a second end to the distal portion of the sheath, wherein the pull wire is configured to transfer at least a portion of a force applied to the first end to the distal portion of the sheath, and wherein the sheath is configured to tear along the one or more structural weaknesses in response to the portion of the force.

Example 23. The method of examples 21 or 22, wherein the one or more structural weaknesses include a plurality of perforations along a length of the distal portion,

Example 24. The method of any of examples 19-23, wherein the implant site includes an anterior mediastinum of the patient, a posterior mediastinum of the patient, or a pleural cavity of the patient.

Example 25. The method of any of examples 19-24, wherein the one or more fixation mechanisms include one or more electrodes, the method further including delivering cardiac pacing to the heart of the patient via the one or more electrodes.

Example 26. The method of any of examples 19-25, wherein the one or more fixation mechanisms includes a first fixation mechanism and a second fixation mechanism, wherein the one or more apertures includes a first aperture and a second aperture, and wherein deploying the one or more fixation mechanisms includes: deploying the first fixation mechanism through the first aperture to engage with the patient tissue at the implant site; and deploying the second fixation mechanism through the second aperture to engage with the patient tissue at the implant site.

Example 27. The method of any of examples 19-26, wherein deploying the one or more fixation mechanisms includes deploying the one or more fixation mechanisms to engage with patient tissue at an extracardiac location.

Example 28. The method of any of examples 19-27, wherein deploying the one or more fixation mechanisms includes deploying the one or more fixation mechanisms to engage with patient tissue at a substernal location.

Example 29. The method of any of examples 19-28, wherein deploying the one or more fixation mechanisms includes deploying the one or more fixation mechanisms to engage with patient tissue at an anterior mediastinum location.

Example 30. The method of any of examples 19-29, wherein the implant site includes a subcutaneous location or a parasternal location.

It will be appreciated by persons skilled in the art that the present application is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the application, which is limited only by the following claims.

What is claimed is:

1. A system comprising:
a sheath comprising an elongated shaft defining a lumen, wherein the sheath comprises:
a distal portion comprising one or more apertures in the shaft; and
an implantable medical lead configured to be delivered through the sheath to an implant site in a patient, wherein the lead comprises:
one or more fixation mechanisms, wherein the one or more fixation mechanisms, when deployed, are configured to extend through the one or more apertures in the sheath and engage with patient tissue at the implant site.

2. The system of claim 1, wherein the distal portion defines a length, and comprises one or more structural weaknesses along the length configured to yield in response to a force.

3. The system of claim 2, wherein the force is a first force, and wherein the system further comprises:
a hub;
a pull wire comprising:
a first end configured to be disposed outside the patient when the sheath is fully inserted in the patient; and
a second end attached to the distal portion of the sheath,
wherein the pull wire is configured to transfer at least a portion of a second force applied to the pull wire at the first end to the distal portion of the sheath as the first force, and
wherein the sheath is configured to tear along the one or more structural weaknesses in response to the first force.

4. The system of claim 2, wherein the one or more structural weaknesses form a path along the length of the distal portion of the sheath that intersects with each of the one or more apertures.

5. The system of claim 2, wherein the one or more structural weaknesses are disposed on the length of the distal portion of the sheath collinear with the one or more apertures.

6. The system of claim 2, wherein the one or more structural weaknesses comprise one or more perforations in the distal portion of the sheath.

7. The system of claim 1,
wherein the one or more apertures are spaced along the length of the distal portion such that when a first aperture of the one or more apertures is positioned at the implant site, a second aperture of the one or more apertures is positioned at the implant site,
wherein the one or more fixation mechanisms includes a first fixation mechanism and a second fixation mechanism,
wherein the first fixation mechanism, when deployed, is configured to extend through the first aperture and engage with the patient tissue at the implant site, and
wherein the second fixation mechanism, when deployed, is configured to extend through the second aperture and engage with the patient tissue at the implant site.

8. The system of claim 1, wherein the implant site comprises an extracardiac, substernal location.

9. The system of claim 1, further comprising an implantable medical device (IMD) configured to be coupled to the lead,
wherein the one or more fixation mechanisms comprise one or more electrodes, and
wherein the IMD is configured to deliver cardiac pacing via the one or more electrodes.

10. The system of any of claim 1, wherein the one or more fixation mechanisms, when deployed, are configured to:
extend through the one or more apertures in the sheath;
engage with the patient tissue while the lead remains inside the sheath; and
pull the lead closer to the implant site.

11. The system of claim 1, wherein the one or more apertures comprise a first aperture and a second aperture, and wherein the first aperture and second aperture are collinear along the length of the distal portion of the sheath.

12. The system of claim 1, wherein the one or more apertures comprise a first aperture and a second aperture, and wherein the first aperture and second aperture are noncollinear along the length of the distal portion of the sheath.

13. The system of claim 1, wherein the one or more fixation mechanisms comprise one or more of side helices, rigid tines, prongs, barbs, clips, screws, disks, pliant tines, flaps, porous structures, metallic or non-metallic scaffolds, or bio-adhesive surfaces.

14. A method comprising:
guiding a lead of a medical device system through a sheath of the medical device system from an incision to an implant site within a patient, wherein the sheath comprises:
an elongated shaft defining a lumen; and
a distal portion comprising one or more apertures in the shaft,
wherein the lead comprises one or more fixation mechanisms, and wherein the one or more fixation mechanisms, when deployed, are configured to extend through the one or more apertures in the sheath;
pressing the sheath toward the heart of the patient;
deploying the one or more fixation mechanisms through the one or more apertures to engage with patient tissue at the implant site and secure the lead in the tissue; and
removing the sheath while retaining the lead.

15. The method of claim 14, further comprising:
creating a tunneling path in a substernal location of the patient via a tunneling rod and the sheath of a medical device system; and
removing the tunneling rod from the tunneling path while retaining the sheath within the tunneling path.

16. The method of claim 14, wherein removing the sheath comprises splitting the distal portion of the sheath along one or more structural weaknesses within the distal portion.

17. The method of claim 16, wherein splitting the distal portion of the sheath along the one or more structural weaknesses comprises applying a force to a first end of a pull wire of the medical device system,
wherein the first end is configured to be disposed outside the patient when the sheath is fully inserted in the patient,
wherein the pull wire is attached at a second end to the distal portion of the sheath,
wherein the pull wire is configured to transfer at least a portion of a force applied to the first end to the distal portion of the sheath, and
wherein the sheath is configured to tear along the one or more structural weaknesses in response to the portion of the force.

18. The method of claim 14, wherein the one or more fixation mechanisms comprise one or more electrodes, the method further comprising delivering cardiac pacing to the heart of the patient via the one or more electrodes.

19. The method of claim 14, wherein the one or more fixation mechanisms comprises a first fixation mechanism and a second fixation mechanism, wherein the one or more apertures comprises a first aperture and a second aperture, and wherein deploying the one or more fixation mechanisms comprises:
deploying the first fixation mechanism through the first aperture to engage with the patient tissue at the implant site; and
deploying the second fixation mechanism through the second aperture to engage with the patient tissue at the implant site.

20. The method of claim 14, wherein deploying the one or more fixation mechanisms comprises deploying the one or more fixation mechanisms to engage with patient tissue at an extracardiac location.
